(19) United States
(12) Patent Application Publication
(10) Pub. No.: US 2021/0134435 A1
(43) Pub. Date: May 6, 2021

(54) BEHAVIOR MODIFICATION ASSISTANCE DEVICE, TERMINAL, AND SERVER

(71) Applicant: OMRON HEALTHCARE Co., Ltd., Kyoto (JP)

(72) Inventors: Toru DENO, Kyoto (JP); Hironori SATO, Kyoto (JP); Takahiro HAMAGUCHI, Kyoto (JP); Takanobu YAMAUCHI, Kyoto (JP); Yasushi MATSUOKA, Kyoto (JP)

(21) Appl. No.: 17/144,406

(22) Filed: Jan. 8, 2021

Related U.S. Application Data
(63) Continuation of application No. PCT/JP2019/026063, filed on Jul. 1, 2019.

(30) Foreign Application Priority Data
Jul. 13, 2018 (JP) 2018-133336

(51) Int. Cl. G16H 20/70; G16H 80/00; G16H 50/30; G16H 20/30; G16H 10/60

(52) U.S. Cl. CPC G16H 20/70; G16H 80/00; G16H 10/60; G16H 20/30; G16H 50/30

(57) ABSTRACT

A behavior modification assistance device includes an acquisition unit acquiring biological data and behavioral data regarding a user, a biological indicator generation unit generating a biological indicator of the user based on the biological data, a behavioral indicator generation unit generating, based on the behavioral data, a behavioral indicator representing an achievement of a behavior by the user affecting the biological indicator, a calculation unit calculating, based on the biological indicator and the behavioral indicator, a requirement level representing, at two or more levels, a need for the user to modify behavior, a determination unit determining, based on the requirement level, an instructor who provides guidance and encourages the user to modify behavior, from among a plurality of candidates including at least a person other than the user, and a guidance request generation unit generating guidance request data requesting the instructor to provide the guidance.

### CROSS-REFERENCE TO RELATED APPLICATIONS

This application is the U.S. national stage application filed pursuant to 35 U.S.C. 365(c) and 120 as a continuation of International Patent Application No. PCT/JP2019/026063, filed Jul. 1, 2019, which application claims priority from Japanese Patent Application No. 2018-133336, Jul. 13, 2018, which applications are incorporated herein by reference in their entireties.

### TECHNICAL FIELD

The present invention relates to a behavior modification assistance device, a terminal, a server, and a non-transitory recording medium in which a program is recorded, for example.

### BACKGROUND ART

In the related art, a physician, a public health nurse, or the like may provide health guidance to patients with various diseases including lifestyle diseases or to potential patients (subjects), the health guidance including, for example, encouraging behavior modification for the lifestyle. However, since the subject is an actual human being, continuing to faithfully follow the guidance received is not easy. For example, even if the subject attempts to improve exercise habits and dietary life immediately after receiving the health guidance, motivation may lower as time passes.

Patent Document 1 discloses simple and easy determination of an optimal degree of intervention in health management of a subject to select an optimal intervention program based on information related to the age, gender, height, weight, waist circumference, obesity, blood pressure, blood glucose level, health psychology assessment measure, and gene polymorphism of a subject who is a diabetes sufferer or who is likely to become diabetic in the future.

### CITATION LIST

### Patent Document

### Patent Document 1: JP 2008-226166 A

### SUMMARY OF INVENTION

According to the technology described in Patent Document 1, the intervention program is selected regardless of how faithfully the subject follows the health guidance. In addition, Patent Document 1 does not describe reviewing the selected intervention program. Thus, the intervention program does not change depending on the actual condition of the subject, that is, whether the subject actively follows or ignores the health guidance. In a case where an intervention program with weekly telephone intervention is maintained for a subject who actively follows the health guidance, more burdens than necessary may be placed on the instructor. On the other hand, in a case where an intervention program with no telephone intervention over an extended period of time is maintained for a subject who ignores the health guidance, the health state of the subject may remain unimproved over an extended period of time or even further deteriorate.

In one aspect, an object of the present invention is to make an approach adapted for the actual condition of the user and to encourage the user to modify behavior.

A behavior modification assistance device according to a first aspect of the present invention includes: an acquisition unit acquiring biological data and behavioral data regarding a user; a biological indicator generation unit generating a biological indicator of the user based on the biological data; a behavioral indicator generation unit generating, based on the behavioral data, a behavioral indicator representing an achievement of a behavior by the user affecting the biological indicator; a calculation unit calculating, based on the biological indicator and the behavioral indicator, a requirement level representing, at two or more levels, a need for the user to modify behavior; a determination unit determining, based on the requirement level, an instructor who provides guidance and encourages the user to modify behavior, from among a plurality of candidates including at least a person other than the user; and a guidance request generation unit generating guidance request data requesting the instructor to provide the guidance.

With the behavior modification assistance device, an instructor of an appropriate level can be selected according to the achievement of behavior affecting the biological indicator, e.g., depending on whether the user actively improves the behavior or not, and the user can be encouraged to modify behavior.

In the behavior modification assistance device according to the first aspect of the present invention, the plurality of candidates may include a first candidate corresponding to a terminal of the user or a machine connected to the terminal; and a second candidate corresponding to a person other than the user, and the determination unit may determine the first candidate as the instructor in a case where the requirement level is a first level and may determine the second candidate as the instructor in a case where the requirement level is a second level higher than the first level.

With the behavior modification assistance device (hereinafter referred to as a behavior modification assistance device according to a second aspect of the present invention), the machine is caused to provide guidance while the requirement level is low, allowing the user to be encouraged to modify behavior without burdening the people around the user. Then, when the behavioral indicator of the user is a poor and a situation continues where the user disregards the guidance of the machine with the behavioral indicator indicating no improvement, the instructor can be switched from the machine to an actual human being other than the user.

In the behavior modification assistance device according to the second aspect, the second candidate may include a person included in a family and acquaintances of the user and selected by the user, the plurality of candidates further include a third candidate corresponding to a medical expert responsible for health guidance for the user or a trainer, and the determination unit may determine the third candidate as the instructor in a case where the requirement level is a third level higher than the second level.

With the behavior modification assistance device (hereinafter referred to as a behavior modification assistance device according to a third aspect of the present invention), in a case where a person other than a user serves as an instructor, a person such as an acquaintance (for example, a friend) or a family member who has opportunities to have daily contact with the user is requested to guide the user, allowing the user to be encouraged to modify behavior within the range of daily life. In a case where this does not stop increasing the requirement level, the instructor is finally switched to a professional such as a medical expert (e.g., a physician, a nurse, a public health nurse, or the like), a trainer, or the like, and the user is more strongly encouraged to modify behavior.

In the behavior modification assistance device according to the third aspect, the calculation unit may set the second level as an upper limit value of the requirement level in a case where the biological indicator is equal to or greater than a first threshold. With the behavior modification assistance device, while the biological indicator indicates a good state even though the behavioral indicator does not indicate a good state, the user can be encouraged to modify behavior within the range of daily life without burdening professionals such as medical experts or a trainers with the need for guidance.

In the behavior modification assistance device according to the third aspect, the calculation unit may set the requirement level to the third level in a case where the biological indicator is less than the first threshold and a last biological indicator is equal to or greater than the first threshold.

With the behavior modification assistance device, in a case where the health state of the user worsens from a good state to a poor state, the instructor can be switched to a professional regardless of the last requirement level, enabling the user to be to immediately strongly encouraged to modify behavior.

In the behavior modification assistance device according to the second or third aspect, the determination unit need not determine the instructor in a case where the requirement level is a fourth level lower than the first level, and the guidance request generation unit need not generate the guidance request data when the instructor is not determined.

With the behavior modification assistance device, the guidance restrains itself in a case where the behavioral indicator of the user indicates a good state or a tendency toward improvement continues, preventing the user from finding the guidance troublesome or from becoming insensitive to the guidance.

In the behavior modification assistance device according to any one of the first to third aspects, the calculation unit need not increase the requirement level compared to a last requirement level in a case where the behavioral indicator is equal to or greater than a second threshold.

With the behavior modification assistance device (hereinafter, referred to as the behavior modification assistance device according to a fourth aspect of the present invention), the requirement level can be maintained or decreased in a case where the achievement of behavior of the user indicates a good state. Decreasing the requirement level allows the instructor to be switched in a step-by-step manner from a professional to a person who has opportunities to have daily contact with the user and then to the machine, reducing burdens on the instructor.

In the behavior modification assistance device according to the fourth aspect, the calculation unit need not decrease the requirement level compared to a last requirement level in a case where the behavioral indicator is less than the second threshold and the behavioral indicator does not indicate an improved state compared to a last behavioral indicator.

With the behavior modification assistance device, the requirement level can be maintained or increased in a case where the achievement of behavior of the user is not good and does not exhibit a tendency toward improvement. Increasing the requirement level allows the instructor to be switched to more strongly encourage the user to modify behavior.

In the behavior modification assistance device according to the fourth aspect, the calculation unit need not increase the requirement level compared to a last requirement level in a case where the behavioral indicator is less than the second threshold and the behavioral indicator indicates an improved state compared to a last behavioral indicator.

With the behavior modification assistance device, in a case where the achievement of behavior of the user does not indicate a good state but a tendency toward improvement, the requirement level can be maintained or decreased. Decreasing the requirement level allows the instructor to be switched in a step-by-step manner from a professional to a person who has opportunities to have daily contact with the user and then to the machine, reducing burdens on the instructor.

In the behavior modification assistance device according to any one of the first to fourth aspects, the behavioral data may be data regarding a plurality of items including at least one of food intake, exercise, medication, sleep, drinking, and smoking of the user, and the guidance request generation unit may generate, based on the behavioral data, guidance request data requesting the guidance for at least one of the plurality of items.

With the behavior modification assistance device, the instructor can provide specific guidance, for example, “Please exercise more,” “Please be sure to take medication,” “Please abstain from drinking/smoking/sugar intake/calorie intake,” and “Please do not stay up late”, and the user easily accepts the guidance.

A terminal according to a fifth aspect of the present invention is a terminal communicating with a server, the terminal including: the behavior modification assistance device according to any one of the first to fourth aspects; and a transmission unit transmitting, in a case where a person other than the user is determined as the instructor, the guidance request data to a terminal of the instructor.

With the terminal, an instructor of an appropriate level can be selected according to the achievement of behavior affecting the biological indicator, e.g., depending on whether the user actively improves behavior or not, and in a case where a person other than the user is determined as the instructor, the guidance request data can be transmitted to the terminal of the instructor to request the instructor to encourage the user to modify behavior.

The terminal according to the fifth aspect may further include: an output data generation unit generating output data encouraging the user to modify behavior based on the guidance request data in a case where the terminal is determined as the instructor; and an output unit outputting the output data.

With the terminal, in a case where the terminal itself is determined as the instructor, for example, output data such as a message text, an image, or voice data indicating a need to improve behavior and/or an improvement-required item can be displayed or audibly output to encourage the user to modify behavior.

In the terminal according to the fifth aspect, in a case where a machine connected to the terminal is determined as the instructor, the transmission unit may transmit the guidance request data to the machine. With the terminal, in a case where the machine connected to the terminal is determined as the instructor, the terminal can transmit the guidance request data to the machine to request the machine to provide an output encouraging the user to modify behavior.

A server according to a sixth aspect of the present invention is a server communicating with a plurality of terminals including a first terminal, the server including: the behavior modification assistance device according to any one of the first to fourth aspects; a reception unit receiving the biological data and the behavioral data from the first terminal; and a transmission unit transmitting the guidance request data to a terminal different from the first terminal in a case where a person other than the user is determined as the instructor.

With the server, an instructor of an appropriate level can be selected according to the achievement of behavior affecting the biological indicator, e.g., depending on whether the user actively improves behavior or not, and in a case where a person other than the user is determined as the instructor, the guidance request data can be transmitted to the terminal of the instructor to request the instructor to encourage the user to modify behavior.

A program recorded in a non-transitory recording medium according to a seventh aspect of the present invention causes a processor to execute processing of each unit included in the behavior modification assistance device according to any one of the first to fourth aspects. With the program, an instructor of an appropriate level can be selected according to the achievement of behavior affecting the biological indicator, e.g., depending on whether the user actively improves behavior or not, and the user can be encouraged to modify behavior.

According to the present invention, an approach adapted for the actual condition of the user can be made to encourage the user to modify behavior.

### BRIEF DESCRIPTION OF DRAWINGS

FIG. 1 is a block diagram illustrating an application example of a terminal according to an embodiment.

FIG. 2 is a diagram illustrating a behavior modification assistance system including the terminal according to the embodiment.

FIG. 3 is a block diagram illustrating a hardware configuration of the terminal according to the embodiment.

FIG. 4 is a block diagram illustrating a functional configuration of the terminal according to the embodiment.

FIG. 5 is a table illustrating an instructor associated with the requirement level for behavior modification.

FIG. 6 is a flowchart illustrating operation of the terminal according to the embodiment.

FIG. 7 is a flowchart illustrating details of step S710 in FIG. 6.

FIG. 8 is a diagram illustrating changes in biological indicator, behavioral indicator, and the requirement level for behavior modification.

FIG. 9 is a block diagram illustrating a functional configuration of a server according to a modified example.

FIG. 10 is a block diagram illustrating a hardware configuration of the server according to the modified example.

### DESCRIPTION OF EMBODIMENTS

An embodiment according to an aspect of the present invention (hereinafter, also referred to as “the present embodiment”) will be described below with reference to the drawings.

Note that elements that are the same as or similar to the elements described hereinafter are given the same or similar reference signs, and duplicate descriptions will be omitted.

### § 1. Application Example

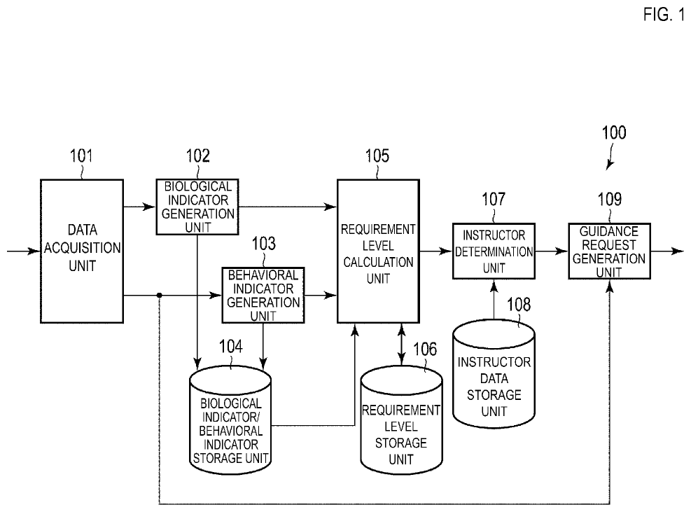

First, an application example of the present embodiment will be described with reference to FIG. 1. FIG. 1 schematically illustrates an application example of a terminal according to the present embodiment. A terminal 100 may be, for example, but not limited to, a mobile terminal (e.g., a smart phone, a tablet, a laptop, a smart watch or other wearable device, or the like), a stationary PC (Personal Computer), or the like.

The terminal 100 calculates the requirement level representing the need for the user to modify behavior, at two or more levels, based on a biological indicator corresponding to the most recent biological data regarding the user that has been indexed; and a behavioral indicator corresponding to the most recent behavioral data regarding the user that has been indexed. The terminal 100 determines, from among the plurality of candidates, an instructor who guides and encourages the user to modify behavior, based on the requirement level, and requests the instructor to guide the user. Note that, focusing on such operation, the terminal 100 may be referred to as a behavior modification assistance device or the like.

As illustrated in FIG. 1, the terminal 100 includes a data acquisition unit 101, a biological indicator generation unit 102, a behavioral indicator generation unit 103, a biological indicator/behavioral indicator storage unit 104, a requirement level calculation unit 105, a requirement level storage unit 106, an instructor determination unit 107, an instructor data storage unit 108, and a guidance request generation unit 109.

The data acquisition unit 101 acquires biological data, which is reference data for evaluation of the health state of the user, and behavioral data regarding the item, of the behavior by the user, which can affect the biological indicator described below (in other words, the item that may be targeted for guidance). The data acquisition unit 101 sends the biological data to the biological indicator generation unit 102 and sends the behavioral data to the behavioral indicator generation unit 103. Note that in a case where guidance request data described below needs to identify an improvement-required item, the data acquisition unit 101 also outputs the behavioral data to the guidance request generation unit 109. The details of the biological data and the behavioral data will be described below.

The biological indicator generation unit 102 receives biological data from the data acquisition unit 101 and generates a biological indicator based on the biological data. Here, the biological indicator may be obtained by executing statistical processing a plurality of biological data or by processing the plurality of biological data, or one most recent biological data may be used directly as the biological indicator. The statistical processing and processing that may be performed on biological data will be described below in detail. The biological indicator generation unit 102 saves the generated biological indicator in the biological indicator/behavioral indicator storage unit 104 and sends the generated biological indicator to the requirement level calculation unit 105.

The behavioral indicator generation unit 103 receives behavioral data from the data acquisition unit 101 and generates a behavioral indicator based on the behavioral data. Here, the behavioral indicator may be obtained by executing statistical processing on a plurality of behavioral data or by processing the plurality of behavioral data, or one most recent behavioral data may be used directly as the behavioral indicator. The statistical processing and processing that may be performed on the behavioral data will be described below in detail. The behavioral indicator generation unit 103 saves the generated behavioral indicator in the biological indicator/behavioral indicator storage unit 104 and sends the generated behavioral indicator to the requirement level calculation unit 105.

The biological indicator/behavioral indicator storage unit 104 stores the biological indicator and the behavioral indicator. The biological indicator is written to the biological indicator/behavioral indicator storage unit 104 by the biological indicator generation unit 102, and the behavioral indicator is written to the biological indicator/behavioral indicator storage unit 104 by the behavioral indicator generation unit 103. The biological indicator and the behavioral indicator are read out from the biological indicator/behavioral indicator storage unit 104 as necessary by the requirement level calculation unit 105.

The requirement level calculation unit 105 receives the current biological indicator from the biological indicator generation unit 102 and receives the current behavioral indicator from the behavioral indicator generation unit 103. The requirement level calculation unit 105 reads out the last biological indicator and behavioral indicator from the biological indicator/behavioral indicator storage unit 104 as necessary and reads out the last requirement level from the requirement level storage unit 106 as necessary.

The requirement level calculation unit 105 calculates the requirement level representing the need for the user to modify behavior, based on the current biological indicator and behavioral indicator and, as necessary, some or all of the last biological indicator, behavioral indicator, and requirement level. The requirement level calculation unit 105 stores the calculated requirement level in the requirement level storage unit 106 and sends the requirement level to the instructor determination unit 107. Note that a specific algorithm for calculating the requirement level will be described below using FIG. 7.

The requirement level storage unit 106 stores the requirement level. The requirement level is written to the requirement level storage unit 106 by the requirement level calculation unit 105 and is read out from the requirement level storage unit 106 as necessary.

The instructor determination unit 107 receives the requirement level from the requirement level calculation unit 105 and reads out instructor data for a plurality of candidates from the instructor data storage unit 108. Here, the plurality of candidates include at least a person other than the user. The instructor determination unit 107 determines the instructor from among the plurality of candidates and sends the instructor data corresponding to the instructor to the guidance request generation unit 109. The instructor data and the candidates for the instructor are described below in detail.

The instructor data storage unit 108 stores the instructor data. The instructor data may be written to the instructor data storage unit 108 based on input from the user or the instructor, for example, when a behavior modification assistance service provided by the terminal 100 is initialized or when settings are changed (a change of the instructor or a change in detailed information regarding the instructor). Additionally, the instructor data is read out from the instructor data storage unit 108 as necessary by the instructor determination unit 107.

The guidance request generation unit 109 receives the instructor data from the instructor determination unit 107 and generates guidance request data for requesting the instructor to guide and encourage the user to modify behavior. The guidance request data will be described below in detail.

As described above, the terminal 100 according to the application example calculates the requirement level representing the need for the user to modify behavior, based on the biological indicator of the user and the behavioral indicator representing the achievement of behavior by the user affecting the biological indicator. The terminal 100 determines, based on the requirement level, the instructor who guides and encourages the user to modify behavior from among a plurality of candidates including a person other than the user, and the terminal 100 requests the determined instructor to provide guidance. Thus, with the terminal 100, the instructor of the appropriate level can be selected according to the achievement of behavior affecting the biological indicator, e.g., depending on whether the user actively improves the behavior or not, and the user can be encouraged to modify behavior.

### § 2. Configuration Example

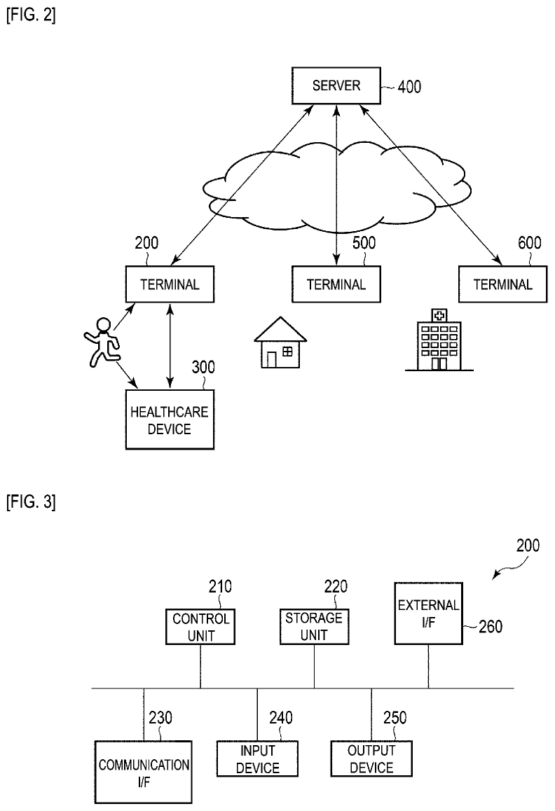

A terminal 200 according to the present embodiment may be included in the behavior modification assistance system illustrated in FIG. 2, for example. The system includes the terminal 200 of the user, a healthcare device 300, a server 400, a terminal 500 of the user's family member as (a candidate of) the instructor, and a terminal 600 of a medical expert (e.g., a physician, a nurse, a public health nurse, or the like) or a trainer for a health management/promotion service provided to the user, the medical expert or the trainer serving as (a candidate of) the instructor.

A hardware configuration and a functional configuration of the terminal 200 will be described below. The healthcare device 300 may be a known healthcare device, such as a blood pressure monitor, a body scale, a blood glucose meter, a pedometer, an activity meter, or the like. The server 400 may be a known server. The terminal 500 and the terminal 600 may also be known terminals.

The terminal 200, the terminal 500, and the terminal 600 are connected to the server 400 via a network such as the Internet, for example, and can exchange data with one another. In other words, the server 400 relays data between the terminals.

The server 400 is not limited to one server, and may be a plurality of different servers. For example, the server 400 may include some or all of a server providing an email or other communication service and a Personal Health Record (PHR) server.

For example, the terminal 200 may transmit the guidance request data to the server 400, which relays the guidance request data to the terminal 500 of the family member as the instructor or to the terminal 600 of the medical expert or trainer as the instructor. Conversely, the terminal 200 may receive for example, an email containing a message encouraging behavior modification, from the terminal 500 or the terminal 600 via the server 400. Furthermore, the terminal 200 may transmit the biological data and/or the biological indicator; and the behavioral data and/or the behavioral indicator to the server 400 for recording as a PHR.

Additionally, the terminal 200 is connected to the healthcare device 300 and can exchange data, via a network such as, for example, a Wireless Local Area Network (WLAN) or using a near-field wireless communication such as Bluetooth (trade name).

Specifically, the terminal 200 may receive the biological data and/or behavioral data from the healthcare device 300. Note that, in a case where the terminal 200 can acquire the necessary biological data and behavioral data, for example, where the terminal 200 includes a biological sensor and a motion sensor, the healthcare device 300 may be unnecessary.

### Hardware Configuration

Now, an example of a hardware configuration of the terminal 200 according to the present embodiment will be described using FIG. 3. FIG. 3 schematically illustrates an example of a hardware configuration of the terminal 200 according to the present embodiment.

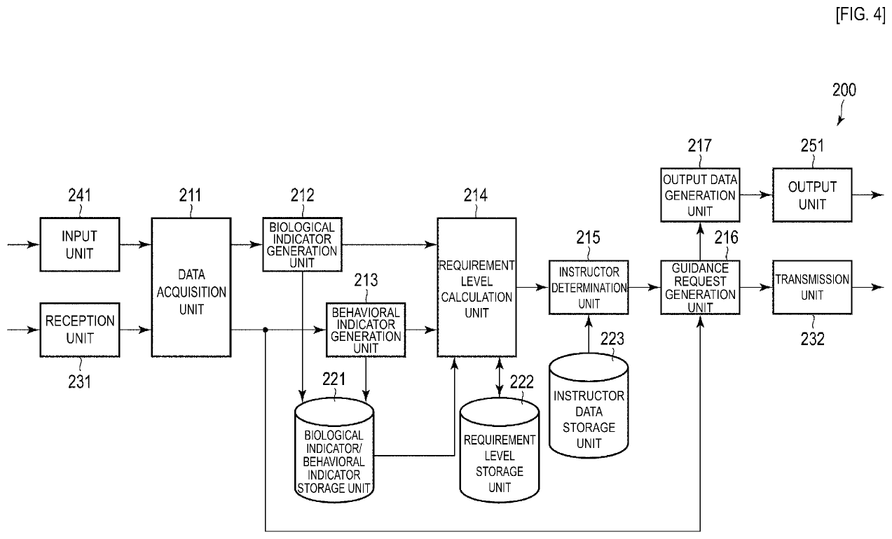

As illustrated in FIG. 4, the terminal 200 according to the present embodiment may be a computer in which a control unit 210, a storage unit 220, a communication I/F 230, an input device 240, an output device 250, and an external I/F 260 are electrically connected, for example, via a bus.

The control unit 210 includes a Central Processing Unit (CPU), a Random Access Memory (RAM), a Read Only Memory (ROM), and the like. The CPU deploys, in the RAM, a program stored in the storage unit 220. The CPU interprets and executes the program to enable the control unit 210 to execute various types of information processing, for example, processing and control of components described in the functional configuration section.

Note that the CPU may be replaced or combined with another processor, for example, a Graphics Processing Unit (GPU), a microcomputer, a Field Programmable Gate Array (FPGA), a Digital Signal Processor (DSP), or the like.

The storage unit 220 is a so-called auxiliary storage device and may be, for example, a Hard Disk Drive (HDD), a Solid State Drive (SSD), or a semiconductor memory such as a flash memory, which is either of a built-in type or of an external type.

The storage unit 220 stores programs executed by the control unit 210 (e.g., a program for causing the control unit 210 to execute behavior modification assistance processing), data used by the control unit 210 (the data may include some or all of, e.g., biological data, behavioral data, biological indicator, behavioral indicator, requirement level, instructor data, guidance request data, and the like), and the like.

The communication I/F 230 may be any of various communication modules for, for example, Bluetooth (including Bluetooth Low Energy), mobile communications (such as 3G and 4G), a LAN (including WLAN), and the like and may be an I/F for performing communications. For example, the communication I/F 230 may include a signal processing circuit, an antenna, a LAN terminals and the like for transmission and reception.

The input device 240 may include a device for accepting user input, such as a touch screen, a keyboard, and a mouse. The input device 240 may also include a sensor that measures a predetermined physical quantity and generates and inputs sensing data (which may include biological data and/or behavioral data). The output device 250 is a device for providing output, for example, a display, a speaker, or the like.

The external I/F 260 is a Universal Serial Bus (USB) port, a memory card slot, or the like and is an I/F for connection with an external device.

The terminal 200 may further include a drive for loading programs and/or data from a storage medium for saving programs and/or data.

Note that, with regard to the specific hardware configuration of the terminal 200, omission, replacement, and addition of a component can be made according to the embodiment as appropriate. For example, the control unit 210 may include a plurality of processors. The terminal 200 may be a general purpose information processing device or a dedicated information processing device designed for services provided. Additionally, the terminal 200 may include a plurality of information processing devices.

### Functional Configuration

Now, an example of a functional configuration of the terminal 200 according to the present embodiment will be described using FIG. 4. FIG. 4 schematically illustrates an example of the functional configuration of the terminal 200.

As illustrated in FIG. 4, the terminal 200 includes a data acquisition unit 211, a biological indicator generation unit 212, a behavioral indicator generation unit 213, a requirement level calculation unit 214, an instructor determination unit 215, a guidance request generation unit 216, an output data generation unit 217, a biological indicator/behavioral indicator storage unit 221, a requirement level storage unit 222, an instructor data storage unit 223, a reception unit 231, a transmission unit 232, an input unit 241, and an output unit 251.

The data acquisition unit 211 may be implemented by the control unit 210 described above, for example. The data acquisition unit 211 acquires the biological data and behavioral data regarding the user from the reception unit 231 and/or the input unit 241 described below. The data acquisition unit 211 sends the biological data to the biological indicator generation unit 212 and sends the behavioral data to the behavioral indicator generation unit 213. Note that, in a case where the guidance request data described below needs to identify an improvement-required item, the data acquisition unit 211 also outputs the behavioral data to the guidance request generation unit 216.

Here, the biological data is reference data for evaluating the health state of a user, such as, for example, blood pressure data, weight data, body fat data, and blood glucose level data. In other words, the user is required through guidance to modify behavior for improved blood pressure, weight, blood glucose level, and the like.

The data acquisition unit 211 may acquire, from the input unit 241, biological data generated by a biological sensor (not illustrated) attached to the terminal 200 or may acquire, from the reception unit 231, biological data received from the healthcare device 300 such as a blood pressure monitor, a body scale, or a blood glucose meter connected with the terminal 200.

On the other hand, the behavioral data may be data regarding the item, of the behavior by the user, which may affect the biological indicator (in other words, the item that may be targeted for guidance), such as exercise, food intake, medication, sleep, drinking, and/or smoking. Specifically, the behavioral data may be data such as the number of steps within a unit period, exercise intensity, activity amount, exercise time, calorie intake, salt content, glucose or fat content, diet contents, medication frequency, sleep time, bedtime, wake-up time, alcohol intake, smoking frequency, and the like.

The data acquisition unit 211 may acquire, from the input unit 241, behavioral data generated by a motion sensor (not illustrated in the drawings, for example, an acceleration sensor, a gyroscope, or the like), may acquire, from the input unit 241, behavioral data generated in the terminal 200 based on self-reporting of the user, or may acquire, from the reception unit 231, behavioral data received from the healthcare device 300 (e.g., a pedometer, an activity meter) connected to the terminal 200.

The biological indicator generation unit 212 may be implemented by the control unit 210 described above, for example. The biological indicator generation unit 212 receives biological data from the data acquisition unit 211 and generates a biological indicator based on the biological data. As described above, the biological indicator may be obtained by executing statistical processing on a plurality of biological data or by processing the plurality of biological data, or one most recent biological data may be used directly as the biological indicator. In the former case, the biological indicator may be generated based on the most recent biological data, e.g., the biological data obtained during the last week or five most recent biological data. The statistical processing may include averaging; or extraction of a minimum, a maximum, a mode, or a median. Additionally, the processing may include normalization, quantization, merging of a plurality of data of different types (e.g., blood pressure data and weight data), and the like. The biological indicator generation unit 212 saves the generated biological indicator in the biological indicator/behavioral indicator storage unit 221 and sends the generated biological indicator to the requirement level calculation unit 214.

The behavioral indicator generation unit 213 may be implemented by the control unit 210 described above, for example. The behavioral indicator generation unit 213 receives behavioral data from the data acquisition unit 211 and generates a behavioral indicator based on the behavioral data. As described above, the behavioral indicator may be obtained by executing statistical processing on a plurality of behavioral data or by processing the plurality of behavioral data, or the most recent behavioral data may be used directly as the behavioral indicator. In the former case, the behavioral indicator may be generated based on the most recent behavioral data, e.g., the behavioral data obtained during the last week or five most recent behavioral data. The statistical processing may include averaging; or extraction of a minimum, a maximum, a mode, or a median. Additionally, processing may include normalization, quantization, merging of a plurality of data of different types (e.g., exercise data and medication data), and the like. The behavioral indicator generation unit 213 saves the generated behavioral indicator in the biological indicator/behavioral indicator storage unit 221 and sends the generated behavioral indicator to the requirement level calculation unit 214.

The requirement level calculation unit 214 may be implemented by the control unit 210 described above, for example. The requirement level calculation unit 214 receives the current biological indicator from the biological indicator generation unit 212 and receives the current behavioral indicator from the behavioral indicator generation unit 213. The requirement level calculation unit 214 reads out the last biological indicator and behavioral indicator from the biological indicator/behavioral indicator storage unit 221 as necessary and reads out the last requirement level from the requirement level storage unit 222 as necessary.

The requirement level calculation unit 214 calculates the requirement level, based on the current biological indicator and the behavioral indicator and, as necessary, some or all of the last biological indicator, behavioral indicator, and requirement level. The requirement level calculation unit 214 stores the calculated requirement level in the requirement level storage unit 222 and sends the requirement level to the instructor determination unit 215. Note that a specific algorithm for calculating the requirement level will be described below using FIG. 7.

The instructor determination unit 215 may be implemented by the control unit 210 described above, for example. The instructor determination unit 215 receives the requirement level from the requirement level calculation unit 214 and reads out instructor data for a plurality of candidates from the instructor data storage unit 223. As described above, the plurality of candidates includes at least a person other than the user. The instructor determination unit 215 determines the instructor from among the plurality of candidates and sends the instructor data corresponding to the determined instructor to the guidance request generation unit 216.

Specifically, the instructor data may include, for each candidate, the requirement level assigned to the candidate, and detailed information regarding the candidate, such as a name, address data, and the like. The instructor determination unit 215 searches the plurality of candidates included in the instructor data for a candidate assigned the requirement level specified by the requirement level calculation unit 214 and determines the instructor.

Additionally, the candidates for the instructor may include, for example, a person responsible for health guidance for the user such as a medical expert (e.g., a physician, a nurse, a public health nurse, or the like) or a trainer for a health management/promotion service provided to the user; and one of people who are linked to the user, such as family members and acquaintances of the user (e.g., friends), the one person being selected by the user; and further the terminal 200 or any other machine, for example, a machine connected to the terminal 200, such as a wearable device, an Artificial Intelligence (AI) speaker, or the healthcare device 300. Determining the instructor from among the plurality of candidates and requesting the instructor to provide guidance, based on the most recent achievement of behavior and the health state of the user, the user can be effectively encouraged to modify behavior.

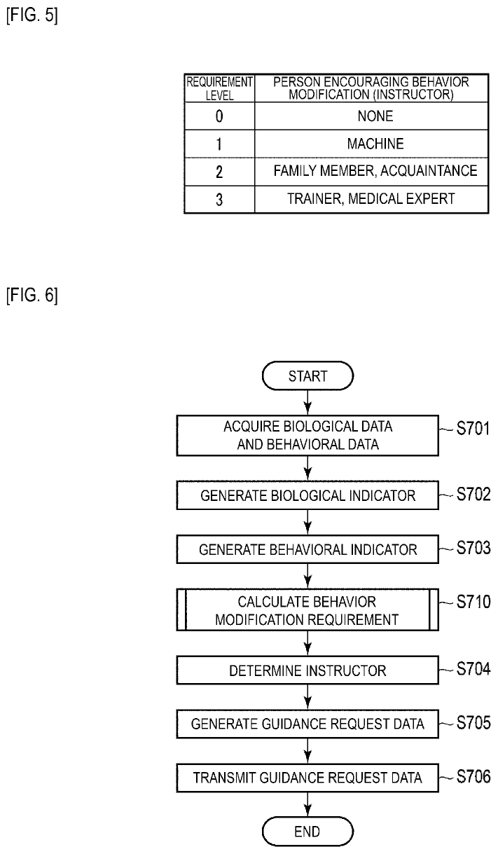

In particular, as illustrated in FIG. 5, a machine may be assigned a low requirement level “1” and a person responsible for health guidance (e.g., a medical expert or a trainer) may be assigned a high requirement level “3,” and any other person (e.g., a family member or an acquaintance) who is linked to the user may be assigned a moderate requirement level “2.”

This allows the machine to guide the user while the requirement level is low, allowing the user to be encouraged to modify behavior without burdening the people around the user. Then, when the behavioral indicator of the user is poor and a situation continues where the user disregards the guidance of the machine with the behavioral indicator indicating no improvement, the requirement level gradually worsens (increases). As the requirement level increases, the instructor is switched from the machine to a person other than the user. First, an acquaintance, a family member, or the like who has opportunities to have daily contact with the user is requested to guide the user, and the user is encouraged to modify behavior within the range of daily life. In a case where this does not stop increasing the requirement level, the instructor is finally switched to a professional such as a medical expert or a trainer, and the user is more strongly encouraged to modify behavior.

Note that, in order to deal with a case where the user's biological indicator and/or behavioral indicator indicates a good state enough to eliminate the need for guidance of the machine, a requirement level “0” that is lower than the requirement level “1” assigned to the machine may be assigned to “no” instructor. Then, the instructor determination unit 215 need not determine the instructor in a case where the requirement level is 0, and the guidance request generation unit 216 need not generate instructor data in a case where no instructor is determined. Thus, in a case where the behavioral indicator of the user indicates a good state or a tendency toward improvement continues, the guidance restrains itself, preventing the user from finding the guidance troublesome or from becoming insensitive to the guidance.

Additionally, the requirement level may be further subdivided and assigned to the instructor. For example, the assigned requirement level may vary among a family member, a friend, and an acquaintance other than friends, or the assigned requirement level may vary among a trainer, a nurse, a public health nurse, and a physician.

The guidance request generation unit 216 may be implemented by the control unit 210 described above, for example. The guidance request generation unit 216 receives the instructor data from the instructor determination unit 215 and generates guidance request data requesting the instructor to guide and encourage the user to modify behavior.

For example, in a case where the instructor is the terminal 200 or any other machine, the guidance may be performed by outputting, in a message text, an image, or a voice, a content (which may include an improvement-required item) to be sent to the user, for example, the need to improve behavior. Accordingly, the guidance request generation unit 216 may generate the message text, the image, or the voice data itself as guidance request data or may generate, as guidance request data, an identifier for identifying such data, the identifier being prepared in advance inside the machine as an instructor or on a cloud. Then, in a case where the instructor is the terminal 200, the data is sent to the output data generation unit 217. On the other hand, in a case where the instructor is a machine other than the terminal 200, the guidance request generation unit 216 sends the guidance request data to the transmission unit 232 of the terminal 200.

In addition, in a case where the instructor is a person other than the user, the guidance may be performed by the instructor sending guidance contents to the user orally (face-to-face communication, phone communication, or the like) or on a text basis (email, Social Networking Service (SNS), or the like). Accordingly, the guidance request generation unit 216 may generate guidance request data including message (text, image, and/or voice) data indicating guidance to be provided to the user and/or a content to be sent to the user (the content including an improvement-required item) or may generate guidance request data including an identifier for identifying such data, the identifier being prepared in advance inside the terminal of the instructor or on a cloud. Furthermore, the guidance request data may include data such as the identifier or name of the user to enable the instructor to identify who is the guidance subject. The guidance request generation unit 216 sends the guidance request data to the transmission unit 232, and the guidance request data reaches the terminal of the instructor, such as the terminal 500 or the terminal 600, via the server 400. The instructor can learn, through the message, the guidance to be provided to the user and/or the content to be sent to the user and can guide the user.

Furthermore, the guidance request generation unit 216 may receive the behavioral data from the data acquisition unit 211, may identify, based on the behavioral data, an improvement-required item from the behavior of the user which needs to be improved, and may generate guidance request data requesting guidance for the requesting item. Thus, the instructor can provide specific guidance, for example, “Please exercise more,” “Please be sure to take medication,” “Please abstain from drinking/smoking/sugar intake/calorie intake,” and “Please do not stay up late”, and the user easily accepts the guidance.

The output data generation unit 217 may be implemented by the control unit 210 described above, for example. The output data generation unit 217 receives the guidance request data from the guidance request generation unit 216, generates, based on the guidance request data, output data encouraging the user to modify behavior, and sends the generated output data to the output unit 251. The output data may be, for example, a message text, an image, or voice data indicating the need to improve behavior and/or an improvement-required item. For example, in a case where the guidance request data is the identifier for identifying the output data, the identifier being prepared inside the terminal 200 as an instructor or on the cloud, then the output data generation unit 217 may acquire output data based on the identifier. Note that, in a case where the guidance request data is directly available as the output data, the output data generation unit 217 may be unnecessary.

The biological indicator/behavioral indicator storage unit 221 may be implemented by the RAM in the control unit 210 described above and/or the storage unit 220. The biological indicator/behavioral indicator storage unit 221 stores the biological indicator and the behavioral indicator. The biological indicator is written to the biological indicator/behavioral indicator storage unit 221 by the biological indicator generation unit 212, and the behavioral indicator is written to the biological indicator/behavioral indicator storage unit 221 by the behavioral indicator generation unit 213. The biological indicator and the behavioral indicator are read out from the biological indicator/behavioral indicator storage unit 221 by the requirement level calculation unit 214.

The requirement level storage unit 222 may be implemented by the RAM in the control unit 210 described above and/or the storage unit 220. The requirement level storage unit 222 saves the requirement level. The requirement level is written to the requirement level storage unit 222 by the requirement level calculation unit 214 and is read out from the requirement level storage unit 222 as necessary.

The instructor data storage unit 223 may be implemented by the RAM in the control unit 210 described above and/or the storage unit 220. The instructor data storage unit 223 stores the instructor data. The instructor data may be written to the instructor data storage unit 223 based on input from the user or the instructor, for example, when a behavior modification assistance service provided by the terminal 200 is initialized or when settings are changed (a change of the instructor or a change in detailed information regarding the instructor). Additionally, the instructor data is read out from the instructor data storage unit 223 as necessary by the instructor determination unit 215.

The reception unit 231 may be implemented by the above-described communication I/F 230. The reception unit 231 receives various data from an external device. For example, the reception unit 231 may receive biological data and/or behavioral data from the healthcare device 300. The reception unit 231 sends the received data to the data acquisition unit 211.

The transmission unit 232 may be implemented by the above-described communication I/F 230. The transmission unit 232 transmits various data to the external device. For example, the transmission unit 232 may receive the guidance request data from the guidance request generation unit 216 and transmit the guidance request data to the appropriate destination, for example, the healthcare device 300, the terminal 500, or the terminal 600. Note that the destination of the guidance request data may be identified, for example, by address data included in the guidance request data. Additionally, in the system in FIG. 2, the guidance request data may be transmitted to the terminal 500 or the terminal 600 via the server 400.

The input unit 241 may be implemented by the input device 240 and/or the external I/F 260 described above. The input unit 241 receives various data. Specifically, the input unit 241 may receive sensing data as the biological data and/or the behavioral data from sensors external to the terminal 200, may perform observation as a sensor built in the terminal 200 to generate biological data and/or behavioral data, or may generate biological data and/or behavioral data based on user input (self-reporting) to the terminal 200. The input unit 241 sends the biological data and/or the behavioral data to the data acquisition unit 211.

The output unit 251 may be implemented by the output device 250 and/or the external I/F 260 described above. The output unit 251 outputs various data. Specifically, the output unit 251 may accept output data from the output data generation unit 217 and output the output data. In other words, the output unit 251 may display/audibly output the output data or may output the output data to an output device external to the terminal 200.

### § 3. Operation Example

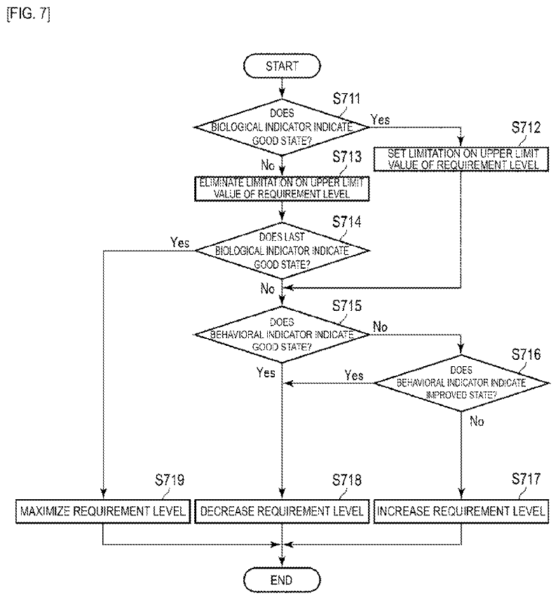

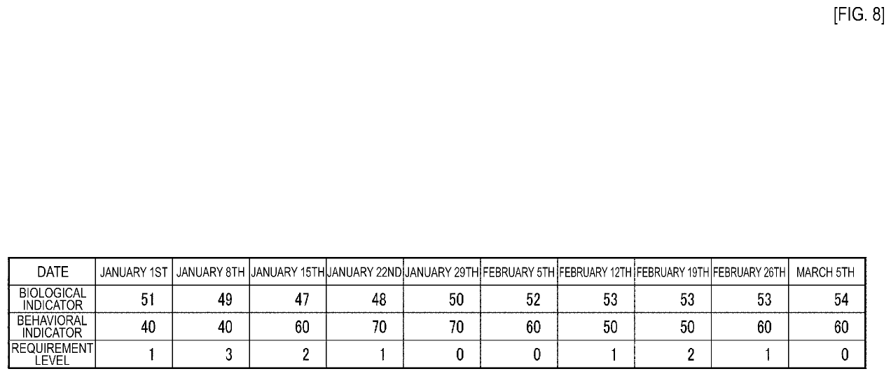

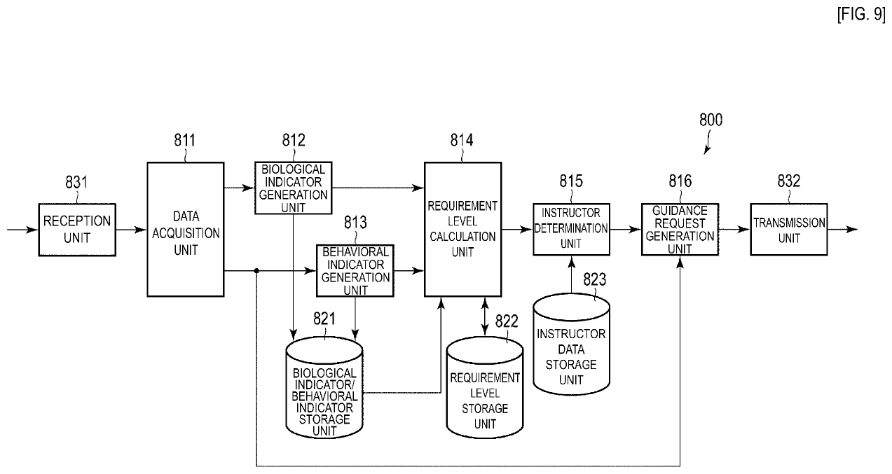

Now, an example of operation of the terminal 200 will be described using FIG. 6 or FIG. 8. Note that the processing procedure described below is merely an example, and each process may be changed to the extent possible. Further, in the processing procedure described below, steps can be omitted, substituted, and added in accordance with the embodiment as appropriate.

FIG. 6 is a flowchart illustrating an example of operation of the terminal 200. First, the data acquisition unit 211 acquires the biological data and the behavioral data from the reception unit 231 and/or the input unit 241 (step S701).

Then, the biological indicator generation unit 212 generates a biological indicator based on the biological data acquired in step S701 (step S702). On the other hand, the behavioral indicator generation unit 213 generates a behavioral indicator based on the behavioral data acquired in step S701 (step S703). Note that the steps S702 and S703 may be performed in an order reverse to the order in FIG. 6 or may be performed in parallel.

The requirement level calculation unit 214 calculates a requirement level representing the need for the user to modify behavior based on the biological indicator and the behavioral indicator generated in step S702 and step S703, respectively (step S710). Step S710 will be described below in detail using FIG. 7.

The instructor determination unit 215 determines, based on the requirement level calculated in step S710, an instructor who guides and encourages the user to modify behavior (step S704). The guidance request generation unit 216 generates guidance request data for requesting the instructor determined in step S704 to guide and encourage the user to modify behavior (step S705).

Then, the transmission unit 232 transmits the guidance request data generated in step S705 (step S706), and the operation in FIG. 6 ends. Note that, as described above, in a case where the instructor is the terminal 200, instead of step S706, operation is performed in which the output data generation unit 217 generates output data based on the guidance request data generated in step S705 and in which the output unit 251 outputs the output data.

Now, step S710 will be described in detail using FIG. 7.

First, the requirement level calculation unit 214 determines whether the (current) biological indicator generated in step S702 indicates a good state (step S711). In a case where the biological indicator is determined to indicate a good state, the processing proceeds to step S712, and in a case where the biological indicator is determined not to indicate a good state, the processing proceeds to step S713.

For example, in a case where the biological indicator is a numerical value increasing with improvement in the health state of the user, the requirement level calculation unit 214 may compare the biological indicator with a predetermined first threshold, may determine the biological indicator to indicate a good state in a case where the biological indicator is equal to or greater than the first threshold, and may determine the biological indicator not to indicate a good state in a case where the biological indicator is below the first threshold.

In step S712, the requirement level calculation unit 214 sets the limitation on the upper limit value of the requirement level, and the processing proceeds to step S715. For example, the requirement level calculation unit 214 may limit the upper limit value to the requirement level corresponding to “2” in FIG. 5. Thus, while the biological indicator indicates a good state even though the behavioral indicator does not indicate a good state, the user can be encouraged to modify behavior within the range of daily life without placing guidance burdens on the professional such as a medical expert or a trainer.

On the other hand, in step S713, the requirement level calculation unit 214 eliminates the limitation on the upper limit value of the requirement level. Then, the requirement level calculation unit 214 reads out the last biological indicator from the biological indicator/behavioral indicator storage unit 221 and determines whether the last biological indicator indicates a good state (step S714).

In a case where, in step S714, the last biological indicator is determined to indicate a good state, this means that the health state has turned (worsened) from a good state to a poor state. In this case, the processing proceeds to step S719. On the other hand, in a case where, in step S714, the last biological indicator is determined not to indicate a good state, this means that the health state of the user is continuously not good. In this case, the processing proceeds to step S715.

In step S715, the requirement level calculation unit 214 determines whether the (current) behavioral indicator generated in step S703 indicates a good state. In a case where the behavioral indicator is determined to indicate a good state, the processing proceeds to step S718, and in a case where the behavioral indicator is determined to not to indicate a good state, the processing proceeds to step S716.

For example, in a case where the behavioral indicator is a numerical value increasing with improvement in achievement of the user's behavior, the requirement level calculation unit 214 may compare the behavioral indicator with a predetermined second threshold, may determine the behavioral indicator to indicate a good state in a case where the behavioral indicator is equal to or greater than the second threshold, and may determine the behavioral indicator not to indicate a good state in a case where the behavioral indicator is below the second threshold.

In step S716, the requirement level calculation unit 214 reads out the last behavioral indicator from the biological indicator/behavioral indicator storage unit 221 and determines whether the behavioral indicator indicates an improved state. In a case where the behavioral indicator is determined to indicate an improved state, the processing proceeds to step S718, and in a case where the behavioral indicator is determined not to indicate an improved state, the processing proceeds to step S717.

For example, in a case where the behavioral indicator is a numerical value increasing with improvement in achievement of the user's behavior, the requirement level calculation unit 214 may compare the current behavioral indicator with the last behavioral indicator, may determine the behavioral indicator to indicate an improved state in a case where the current behavioral indicator is greater than the last behavioral indicator, and may determine the behavioral indicator not to indicate an improved state in a case where the current behavioral indicator is less than or equal to the last behavioral indicator. Additionally, comparison with the last behavioral indicator is not necessarily required, and comparison may be made with, for example, a statistic (e.g., mean, median, maximum, minimum, or mode) of a plurality of recent behavioral indicators.

In step S717, the requirement level calculation unit 214 reads out the last requirement level from the requirement level storage unit 222, increases the read-out requirement level, and writes the requirement level to the requirement level storage unit 222, and the processing in step S710 ends. Thus, in a case where the achievement of behavior of the user is not in a good state and does not exhibit a tendency toward improvement, the requirement level is increased and the instructor is switched, enabling the user to be more strongly encouraged to modify behavior.

The increment in step S717 may be “1” or may be a different value different from “1.” In addition, instead of increasing the requirement level each time step S717 is executed, it is possible to increase the requirement level each time step S717 is executed twice or more times. In any case, in step S717, the requirement level calculation unit 214 calculates the requirement level at least not to be decreased than the last requirement level.

In step S718, the requirement level calculation unit 214 reads out the last requirement level from the requirement level storage unit 222, decreases the read-out requirement level, and writes the decreased requirement level to the requirement level storage unit 222, and the processing in step S710 ends. Thus, in a case where the achievement of behavior of the user indicates a good state or a tendency toward improvement, the requirement level is decreased and the instructor is switched from a professional to a person who has opportunities to have daily contact with the user and to a machine, enabling a reduction in burden on the instructor.

The decrement in step S718 may be “1” or may be a value different from “1.” In addition, instead of decreasing the requirement level each time step S718 is executed, it is possible to decrease the requirement level each time step S718 is executed twice or more times. In any case, in step S718, the requirement level calculation unit 214 calculates the requirement level at least not to be increased than the last requirement level.

In step S719, the requirement level calculation unit 214 writes the maximum value of the requirement level to the requirement level storage unit 222, and the processing in step S710 ends. Thus, in a case where the health state of the users worsens from a good state to a poor state, the instructor is switched to the professional regardless of the last requirement level, enabling the user to be immediately strongly encouraged to modify behavior.

Note that, in step S719, the requirement level calculation unit 214 need not necessarily maximize the requirement level. For example, as in step S717, the requirement level calculation unit 214 may calculate the requirement level to be increased compared to the last requirement level. The increment in step S719 may be determined to be greater than the increment in step S717.

The following is a description of an example of changes in biological indicator, behavioral indicator, and requirement level using FIG. 8. In the example of FIG. 8, the biological indicator/behavioral indicator has a numerical value normalized within a range from “0” to “100” and increasing with improvement in the user's health state/behavior achievement. Additionally, both the first threshold and the second threshold described above are “50”. In addition, the calculation period of the requirement level is one week, and the instructor associated with the requirement level is determined according to the table in FIG. 5.

The biological indicator and behavioral indicator of the user indicate “51” and “40”, respectively, as of January 1st and the requirement level is calculated to be “1”. Thus, the user is guided by the terminal 200, the healthcare device 300, or any other machine.

The biological indicator and behavioral indicator of the user indicate “49” and “40”, respectively, as of January 8th. Since the biological indicator is determined not to indicate a good state and the last biological indicator is determined to indicate a good state, step S719 is performed and the requirement level is maximized to “3.” Thus, the user is strongly encouraged to modify behavior, for example, under the guidance of a physician (or a public health nurse, a nurse, a trainer).

Successful guidance of the physician significantly increases the behavioral indicator of the user to “60” as of January 15th, which is considered to be good. Accordingly, step S718 is performed and the requirement level decreases to “2”. As a result, the instructor of the user is switched to a family member (or the acquaintance), and the physician is freed of the guidance burden.

As a result of the familial guidance, the behavioral indicator of the user significantly increases to “70” as of January 22. Accordingly, step S718 is performed and the requirement level decreases to “1”. As a result, the instructor of the user is switched to a machine again and the family member is freed of the guidance burden.

As a result of the machine intervention, the user's behavioral indicator is maintained “70”, which is considered to be good. Accordingly, step S718 is performed, and the requirement level decreases to “0,” leading to no guidance to the user.

As a result of no guidance to the user, the behavioral indicator of the user is dropped to “60” as of February 5th. Since the behavioral indicator of “60” is determined to indicate a good state, step S718 is performed and the requirement level remains at the minimum value of “0”. This results in still no guidance to the user.

As a result of no guidance to the user again, the behavioral indicator of the user is dropped to “50” as of February 12th. Since the behavioral indicator of “50” is determined not to indicate a good state, step S719 is performed and the requirement level increases to “1.” As a result, the instructor of the user is determined to be the machine and the guidance is restarted.

Despite the guidance of the machine, the behavioral indicator of the user has not increased from “50”. as of February 19th. Since the behavioral indicator of “50” is determined not to indicate a good state, step S719 is performed and the requirement level is increased to “2”. As a result, the instructor of the user is switched to the family member, and the user is more strongly encouraged to modify behavior.

Successful familial guidance increased the behavioral indicator of the user to “60” as of February 26th, which is considered to be good. Accordingly, step S718 is performed and the requirement level decreases to “1”. As a result, the instructor of the user is switched to the machine again and the family member is freed of the guidance burden.

As a result of the guidance of the machine, the behavioral indicator of the user is maintained at “60”, which is considered to be good. Accordingly, step S718 is performed, and the requirement level decreases to “0,” leading to no guidance to the user again.

As described above, the terminal according to the present embodiment calculates the requirement level representing the need for the user to modify behavior, based on the biological indicator of the user and the behavioral indicator representing the achievement of the behavior by the user affecting the biological indicator. The terminal determines, based on the requirement level, the instructor who guides and encourages the user to modify behavior from among a plurality of candidates including a person other than the user, and the terminal requests the determined instructor to provide guidance. Thus, with the terminal, the instructor of the appropriate level can be selected according to the achievement of behavior affecting the biological indicator, for example, depending on whether the achievement of the behavior indicates a good state or not or, even in a case where the achievement of the behavior does not indicate a good state, whether the achievement indicates a tendency toward improvement or not, and the user can be encouraged to modify behavior. In other words, it can be appropriately switched, for example, whether to leave the behavior of the user to the free will of the user (no guidance), whether to motivate the user through message output by the machine or the like (the machine is the instructor), whether to request another person to motivate the user through talking within the range of daily life (a family member, an acquaintance (e.g., a friend), or the like is the instructor), and whether to request a professional to provide health guidance (a medical expert (e.g., a physician, a public health nurse, a nurse, or the like), a trainer, or the like is the instructor).

### § 4. Modified Examples

While embodiments of the present invention have been described in detail above, the foregoing description is merely illustrative of the present invention in all respects. Of course, various modifications and variations can be made without departing from the scope of the present invention. In other words, specific configurations in accordance with an embodiment may be adopted as appropriate at the time of carrying out the present disclosure. Note that although data appearing in the present embodiment will be described using natural language, the data is more specifically designated by pseudo-language, commands, parameters, machine language, and the like that are recognizable by a computer.

For example, the following changes are possible. Note that, in the following, the same reference numerals are used for components that are the same as those of the above-described embodiment, and descriptions thereof are omitted as appropriate. The following modified examples can be combined as appropriate.

In the present embodiment, an example has been described in which the terminal generates the biological indicator and the behavioral indicator, calculates the requirement level, determines the instructor, and generates the guidance request data. However, part or all of the processing may be executed by the server. Note that, focusing on such operation, the server according to the modified example can also be referred to as a behavior modification assistance device or the like.

Specifically, as illustrated in FIG. 9, a server 800 according to this modified example includes a data acquisition unit 811, a biological indicator generation unit 812, a behavioral indicator generation unit 813, a requirement level calculation unit 814, an instructor determination unit 815, a guidance request generation unit 816, a biological indicator/behavioral indicator storage unit 821, a requirement level storage unit 822, an instructor data storage unit 823, a reception unit 831, and a transmission unit 832.

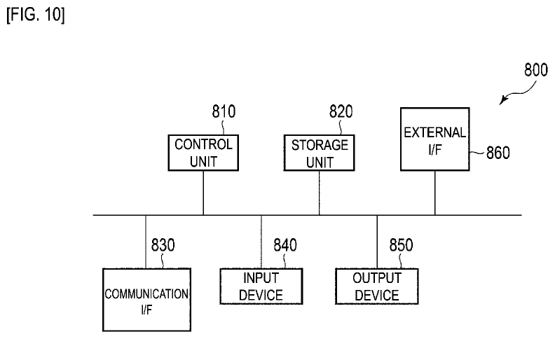

Additionally, the server 800 is a computer, for example, and an example of a hardware configuration of the server 800 is schematically illustrated in FIG. 10. A control unit 810, a storage unit 820, a communication I/F 830, an input device 840, an output device 850, and an external I/F 860 illustrated in FIG. 10 may be respectively basically identical to or similar to the hardware with the identical names illustrated in FIG. 3, without considering difference in specifications. Thus, description of each piece of the hardware illustrated in FIG. 10 is omitted.

Now, the individual components of the server 800 illustrated in FIG. 9 will be described, but descriptions of portions of the server 800 common to components included in the terminal 100 in FIG. 1 or the terminal 200 in FIG. 4 and having identical names may be omitted.

The data acquisition unit 811 acquires the biological data and behavioral data regarding the user from the reception unit 831. Note that, in a case where the server 800 needs to handle data regarding a plurality of users, the data acquisition unit 811 may acquire the identifier of the user from the reception unit 831 in addition to the biological data and the behavioral data.

The biological indicator generation unit 812 and the behavioral indicator generation unit 813 are basically identical to or similar to elements included in the terminal 100 in FIG. 1 or the terminal 200 in FIG. 4 and having identical names. However, in a case where the server 800 needs to handle data regarding a plurality of users, the biological indicator generation unit 812/the behavioral indicator generation unit 813 may associate the identifier of the user with the biological indicator/behavioral indicator.

Similarly, the requirement level calculation unit 814 and the instructor determination unit 815 are basically identical to or similar to elements included in the terminal 100 in FIG. 1 or the terminal 200 in FIG. 4 and having identical names. However, in a case where the server 800 needs to handle data regarding a plurality of users, the requirement level calculation unit 814/the instructor determination unit 815 may associate the identifier of the user with the requirement level/instructor data.

The guidance request generation unit 816 receives instructor data from the instructor determination unit 815, generates, based on the instructor data, guidance request data requesting the instructor to guide and encourage the user to modify behavior, and sends the guidance request data to the transmission unit 832. Note that, in a case where the server 800 needs to handle data regarding a plurality of users, the guidance request generation unit 816 may associate the identifier of the user with the guidance request data.

The reception unit 831 may receive biological data and/or behavioral data from a terminal of the user or a healthcare device, for example. Note that, in a case where the server 800 needs to handle data regarding a plurality of users, the reception unit 831 may receive the identifier of the user in addition to the biological data and the behavioral data.

The transmission unit 832 receives the guidance request data from the guidance request generation unit 216 and may transmit the guidance request data to the appropriate destination, for example, a machine as an instructor (the terminal of the user or the healthcare device); or a terminal of an instructor (a family member, an acquaintance, a medical expert, a trainer, or the like).

In this way, part or all of the processing of the terminal according to the present embodiment can be executed by the server. This has, for example, the advantage of reducing processing loads on the terminal.

### § 5 Feature

A part or the entirety of the embodiment can be described, as described in the following supplementary notes in addition to the scope of the claims, but the present invention is not limited thereto.

A behavior modification assistance device (100) including: an acquisition unit (101) acquiring biological data and behavioral data regarding a user, a biological indicator generation unit (102) generating a biological indicator of the user based on the biological data, a behavioral indicator generation unit (103) generating, based on the behavioral data, a behavioral indicator representing an achievement of a behavior by the user affecting the biological indicator, a calculation unit (105) calculating, based on the biological indicator and the behavioral indicator, a requirement level representing, at two or more levels, a need for the user to modify behavior, a determination unit (107) determining, based on the requirement level, an instructor who provides guidance and encourages the user to modify behavior, from among a plurality of candidates including at least a person other than the user, and a guidance request generation unit (109) generating guidance request data requesting the instructor to provide the guidance.

### REFERENCE NUMBERS

100, 200, 500, 600 . . . Terminal
101, 211, 811 . . . Data acquisition unit
102, 212, 812 . . . Biological indicator generation unit
103, 213, 813 . . . Behavioral indicator generation unit
104, 221, 821 . . . Biological indicator/behavioral indicator storage unit
105, 214, 814 . . . Requirement level calculation unit
106, 222, 822 . . . Requirement level storage unit
107, 215, 815 . . . Instructor determination unit
108, 223, 823 . . . Instructor data storage unit
109, 216, 816 . . . Guidance request generation unit
210, 810 . . . Control unit
217 . . . Output data generation unit
220, 820 Storage unit
230, 830 . . . Communication I/F
231, 831 . . . Reception unit
232, 832 . . . Transmission unit
240, 840 . . . Input device
241 . . . Input unit
250, 850 . . . Output device
251 . . . Output unit
260, 860 . . . External I/F
300 . . . Healthcare device
400, 800 . . . Server

1. A behavior modification assistance device comprising:
an acquisition unit acquiring biological data and behavioral data regarding a user;
a biological indicator generation unit generating a biological indicator of the user based on the biological data;
a behavioral indicator generation unit generating, based on the behavioral data, a behavioral indicator representing an achievement of a behavior by the user affecting the biological indicator;
a calculation unit calculating, based on the biological indicator and the behavioral indicator, a requirement level representing, at two or more levels, a need for the user to modify behavior;
a determination unit determining, as an instructor who provides guidance and encourages the user to modify behavior, one of a plurality of candidates including at least a person other than the user, the one candidate more strongly encouraging the user to modify behavior as the requirement level increases; and
a guidance request generation unit generating guidance request data requesting the instructor to provide the guidance.

2. The behavior modification assistance device according to claim 1, wherein the plurality of candidates include: a first candidate corresponding to a terminal of the user or a machine connected to the terminal; and a second candidate corresponding to a person other than the user, and
the determination unit determines the first candidate as the instructor in a case where the requirement level is a first level and determines the second candidate as the instructor in a case where the requirement level is a second level higher than the first level.

3. The behavior modification assistance device according to claim 2, wherein the second candidate includes a person included in a family or an acquaintance of the user and selected by the user,
the plurality of candidates further include a third candidate corresponding to a medical expert responsible for health guidance for the user or a trainer, and
the determination unit determines the third candidate as the instructor in a case where the requirement level is a third level higher than the second level.

4. The behavior modification assistance device according to claim 3, wherein the calculation unit sets the second level as an upper limit value of the requirement level in a case where the biological indicator is equal to or greater than a first threshold.

5. The behavior modification assistance device according to claim 3, wherein the calculation unit sets the requirement level to the third level in a case where the biological indicator is less than the first threshold and a last biological indicator is equal to or greater than the first threshold.

6. The behavior modification assistance device according to claim 2, wherein the determination unit does not determine the instructor in a case where the requirement level is a fourth level lower than the first level, and
the guidance request generation unit does not generate the guidance request data when the instructor is not determined.

7. The behavior modification assistance device according to claim 1, wherein the calculation unit does not increase the requirement level compared to a last requirement level in a case where the behavioral indicator is equal to or greater than a second threshold.

8. The behavior modification assistance device according to claim 7, wherein the calculation unit does not decrease the requirement level compared to a last requirement level in a case where the behavioral indicator is less than the second threshold and the behavioral indicator does not indicate an improved state compared to a last behavioral indicator.

9. The behavior modification assistance device according to claim 7, wherein the calculation unit does not increase the requirement level compared to a last requirement level in a case where the behavioral indicator is less than the second threshold and the behavioral indicator indicates an improved state compared to a last behavioral indicator.

10. The behavior modification assistance device according to claim 1, wherein the behavioral data is data regarding a plurality of items including at least one of food intake, exercise, medication, sleep, drinking, and smoking of the user, and
the guidance request generation unit generates, based on the behavioral data, guidance request data requesting the guidance for at least one of the plurality of items.

11. A terminal communicating with a server, the terminal comprising:
the behavior modification assistance device according to claim 1; and
a transmission unit transmitting, in a case where a person other than the user is determined as the instructor, the guidance request data to a terminal of the instructor.

12. The terminal according to claim 11, further comprising:
an output data generation unit generating output data encouraging the user to modify behavior based on the guidance request data in a case where the terminal is determined as the instructor; and
an output unit outputting the output data.

13. The terminal according to claim 11, wherein in a case where a machine connected to the terminal is determined as the instructor, the transmission unit transmits the guidance request data to the machine.

14. A server communicating with a plurality of terminals including a first terminal, the server comprising:
the behavior modification assistance device according to claim 1;
a reception unit receiving the biological data and the behavioral data from the first terminal; and
a transmission unit transmitting the guidance request data to a terminal different from the first terminal in a case where a person other than the user is determined as the instructor.

15. A program for causing a processor to execute processing of each unit included in the behavior modification assistance device according to claim 1.

16. The behavior modification assistance device according to claim 1, wherein the determination unit determines a candidate who has more knowledge related to the guidance, as the instructor as the requirement level increases.

17. The behavior modification assistance device according to claim 1, wherein the determination unit determines a candidate who has more expertise, as the instructor as the requirement level increases.

18. The behavior modification assistance device according to claim 3, wherein the determination unit does not determine the instructor in a case where the requirement level is a fourth level lower than the first level, and
the guidance request generation unit does not generate the guidance request data when the instructor is not determined.

19. The behavior modification assistance device according to claim 4, wherein the determination unit does not determine the instructor in a case where the requirement level is a fourth level lower than the first level, and
the guidance request generation unit does not generate the guidance request data when the instructor is not determined.

20. The behavior modification assistance device according to claim 5, wherein the determination unit does not determine the instructor in a case where the requirement level is a fourth level lower than the first level, and
the guidance request generation unit does not generate the guidance request data when the instructor is not determined.
